(19) United States
(12) Patent Application Publication
(10) Pub. No.: US 2025/0054134 A1
(43) Pub. Date: Feb. 13, 2025

(54) SYSTEMS AND METHODS FOR DETECTING OCULAR LESIONS IN FUNDUS IMAGES

(71) Applicant: UTI LIMITED PARTNERSHIP, Calgary (CA)

(72) Inventors: Behrouz Far, Calgary (CA); Trafford Crump, Calgary (CA); Ezekiel Weis, Calgary (CA); Emad A. Mohammed, Kamloops (CA)

(21) Appl. No.: 18/447,213

(22) Filed: Aug. 9, 2023

(51) Int. Cl. G06T 7/00; G06T 3/40; G06T 5/00; G16H 30/40; G16H 50/30

(52) U.S. Cl. CPC G06T 7/0012; G06T 3/40; G06T 5/73; G16H 30/40; G16H 50/30; G06T 2207/20081; G06T 2207/20084; G06T 2207/20132; G06T 2207/30041; G06T 2207/30096

(57) ABSTRACT

Methods and systems are described for detecting ocular lesions indicative of an ocular pathology. The methods and systems involve obtaining a fundus image of an eye of a patient, preprocessing the fundus image to obtain a processed fundus image applying a detection model to the processed fundus image to identify whether a lesion is present in the fundus image providing an output based on an output result of the detection model. The detection model is configured for detecting ocular lesions in fundus images and the output is related to a lesion being present in the fundus image.

### FIELD

Various embodiments are described herein that generally relate to processing medical image data including detecting ocular lesions indicative of ocular pathology in medical images.

### BACKGROUND

Ocular lesions can pose significant risks to the eye health of the individual affected by the lesion(s) and if left untreated, to the overall health of the individual. One common type of ocular lesion is a choroidal nevus. Malignant forms of choroidal nevi can develop into uveal melanoma (UM), a severe intraocular cancer. With an incidence rate of 5.1 cases per million per year in the United States, UM is the most common primary intraocular cancer. If left untreated, UM can lead to vision loss, metastasis, and death. Early identification of UM can allow for early treatment, reducing symptom severity, the risk of death and improving the post-treatment visual outcomes.

A choroidal nevus is a lesion inside and on the back of the eye. Choroidal nevi have an estimated prevalence of 0.2%-30% in adults. Most choroidal nevi are harmless. However, an estimated 0.2% of choroidal nevi transform into UM. Identification and risk of stratifying of choroidal nevi can allow for screening and monitoring for UM.

Existing techniques for identifying and assessing choroidal nevi involve healthcare providers (e.g., primary eye care providers) obtaining fundus images of a patient's eye and visually assessing whether a choroidal nevus is present. When a choroidal nevus is determined to be present and thought to have malignant potential by the healthcare provider, a referral is made to a specialized healthcare provider, such as an ocular oncologist, for diagnosis and treatment. However, visual inspection of fundus images is unreliable, challenging, and can lead to a high rate of false negative and false positive assessments. False negative assessments can cause malignant choroidal nevi to go untreated, leading to increased health risks. False positive assessments can cause healthcare resources to be wasted.

### SUMMARY OF VARIOUS EMBODIMENTS

In a broad aspect, in accordance with the teachings herein, there is provided at least one embodiment of a method for detecting ocular lesions indicative of an ocular pathology. The method comprises: obtaining a fundus image of an eye of a patient; preprocessing the fundus image to obtain a processed fundus image; applying a detection model to the processed fundus image to identify whether an ocular lesion is present in the fundus image, the detection model being configured for detecting the ocular lesion in fundus images; and providing an output based on an output result of the detection model, the output being related to the ocular lesion being present in the fundus image.

In at least one embodiment, when the detection model detects the ocular lesion is present, the detection model is further configured to determine a risk of the ocular lesion being a malignant tumor based on one or more characteristics of the fundus image, and the method further comprises, in response to determining the ocular lesion is a malignant tumor, providing a treatment recommendation based on a predicted risk of growth and/or metastasis, wherein the treatment recommendation includes any combination of a referral recommendation or a suggested treatment, wherein the suggested treatment includes any combination of regular monitoring, radiation therapy, immunotherapy, targeted therapy, or removal of the eye.

In at least one embodiment, the detection model comprises a trained machine learning model including a convolutional neural network (CNN) or a CNN trained via transfer learning.

In at least one embodiment, the CNN trained via transfer learning is one of an Inceptionv3 model, an Xception model, a DensetNet121 model or a DenseNet169 model.

In at least one embodiment, the method further comprises: applying a Shapley Additive explanations (SHAP) analysis to the detection model to determine a contribution of each feature of the fundus image to the output result of the detection model; and displaying a visual representation of the SHAP analysis.

In at least one embodiment, when the detection model detects the ocular lesion is present the method further comprises: annotating the fundus image to obtain an annotated fundus image identifying the ocular lesion; and displaying the annotated fundus image.

In at least one embodiment, when the detection model detects the ocular lesion is present, the method further comprises: resizing the fundus image to obtain an image of the ocular lesion; and displaying the image of the ocular lesion.

In at least one embodiment, the ocular lesion is a choroidal nevus or a uveal melanoma (UM).

In at least one embodiment, preprocessing the fundus image comprises isolating a green channel of the fundus image.

In at least one embodiment, preprocessing the fundus image further comprises one or more of: cropping the fundus image to remove a black portion surrounding a fundus in the fundus image, normalizing the fundus image to reduce light variations and sharpening the fundus image to reduce blurriness.

In another aspect, in accordance with the teachings herein, there is provided at least one embodiment of a system for detecting ocular lesions indicative of an ocular pathology. The system comprises: a database for storing fundus images; a memory for storing software instructions for processing a fundus image; and at least one processor in communication with the memory and the database. The at least one processor, upon executing the software instructions, is configured to: obtain the fundus image of an eye of a patient from the database; preprocess the fundus image to obtain a processed fundus image; apply a detection model to the processed fundus image to identify whether an ocular lesion is present in the fundus image, the detection model being configured for detecting the ocular lesion in fundus images; and provide an output based on an output result of the detection model, the output result being related to the ocular lesion being present in the fundus image.

In at least one embodiment, when the detection model detects the ocular lesion is present, the at least one processor is configured to: use the detection model to determine a risk of the ocular lesion being a malignant tumor based on one or more characteristics of the fundus image; and in response to determining the ocular lesion is malignant, provide a treatment recommendation based on a predicted risk of growth and/or metastasis, wherein the treatment recommendation includes any combination of a referral recommendation or a suggested treatment, wherein the suggested treatment includes any combination of regular monitoring, radiation therapy, immunotherapy, targeted therapy or removal of the eye.

In at least one embodiment, the detection model comprises a trained machine learning model including a convolutional neural network (CNN) or a CNN trained via transfer learning.

In at least one embodiment, the CNN trained via transfer learning is one of an Inceptionv3 model, an Xception model, a DensetNet121 model or a DenseNet169 model.

In at least one embodiment, the at least one processor is further configured to: apply a Shapley Additive explanations (SHAP) analysis to the detection model to calculate a contribution of each feature of the fundus image to the output result of the detection model; and display a visual representation of the SHAP analysis.

In at least one embodiment, when the detection model detects the ocular lesion is present, the at least one processor is configured to: annotate the fundus image to obtain an annotated fundus image identifying the ocular lesion; and display the annotated fundus image.

In at least one embodiment, when the detection model detects the ocular lesion is present, the at least one processor is configured to: resize the fundus image to obtain an image of the ocular lesion; and display the image of the ocular lesion.

In at least one embodiment, preprocessing the fundus image comprises isolating a green channel of the fundus image.

In at least one embodiment, preprocessing the fundus image further comprises one or more of: cropping the fundus image to remove a black portion surrounding a fundus in the fundus image, normalizing the fundus image to reduce light variations and sharpening the fundus image to reduce blurriness.

In another aspect, in accordance with the teachings herein, there is provided at least one embodiment of a non-transitory computer readable medium storing thereon software instructions, which when executed by at least one processor, configure the at least one processor for performing a method for detecting ocular lesions indicative of an ocular pathology wherein the method is defined according to the teachings herein.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating preferred embodiments of the application, are given by way of illustration only since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings, which show at least one example embodiment, and which are now described. The drawings are not intended to limit the scope of the teachings described herein.

FIG. 1 is a block diagram of an example embodiment of a system for detecting ocular lesions indicative of ocular pathology.

FIG. 2A is a fundus image of an eye without ocular lesions.

FIG. 2B is a fundus image of an eye with an ocular lesion.

FIG. 3 is a flowchart of an example embodiment of a method for detecting ocular lesions indicative of ocular pathology.

FIG. 4A is an example fundus image.

FIGS. 4B-4D show the blue, green and red channels, respectively, of the fundus image of FIG. 4A.

FIG. 5 is a diagram showing an architecture of a machine-learning model used for the detection model for detecting ocular lesions.

FIG. 6 shows a visual representation of the operation of a Shapley Additive Explanations Analysis (SHAP).

FIGS. 7A-7B show examples of a fundus image and a visual representation of SHAP analysis, respectively, for an eye found to have no ocular lesions by using a detection model and SHAP analysis according to the teachings herein.

FIGS. 7C-7D show examples of a fundus image and a visual representation of SHAP analysis, respectively, for an eye found to have an ocular lesion by using a detection model and SHAP analysis according to the teachings herein.

FIGS. 8A-8B show examples of a fundus image and a visual representation of SHAP analysis, respectively, for an eye found to have no ocular lesions by using a detection model comprising the InceptionV3 model, trained via transfer learning for detection of ocular lesions and SHAP analysis according to the teachings herein.

FIGS. 8C-8D show examples of a fundus image and a visual representation of SHAP analysis, respectively, for an eye found to an ocular lesion by using a detection model comprising the InceptionV3 model, trained via transfer learning for detection of ocular lesions and SHAP analysis according to the teachings herein.

FIGS. 8E-8F show examples of a fundus image and a visual representation of SHAP analysis, respectively, for an eye found to have no ocular lesions by using a detection model comprising the Xception model, trained via transfer learning for detection of ocular lesions and SHAP analysis according to the teachings herein.

FIGS. 8G-8H show examples of a fundus image and a visual representation of SHAP analysis, respectively, for an eye found to have an ocular lesion by using a detection model comprising the Xception model, trained via transfer learning for detection of ocular lesions and SHAP analysis according to the teachings herein.

FIGS. 8I-8J show examples of a fundus image and a visual representation of SHAP analysis, respectively, for an eye found to have no ocular lesions by using a detection model comprising the DenseNet121 model, trained for detection of ocular lesions via transfer learning and SHAP analysis according to the teachings herein.

FIGS. 8K-8L show examples of a fundus image and a visual representation of SHAP analysis, respectively, for an eye found to have an ocular lesion by using a detection model comprising the DenseNet121 model, trained via transfer learning for detection of ocular lesions and SHAP analysis according to the teachings herein.

FIGS. 8M-8N show examples of a fundus image and a visual representation of SHAP analysis, respectively, for an eye found to have no ocular lesions by using a detection model comprising the DenseNet169 model, trained via transfer learning for detection of ocular lesions and SHAP analysis according to the teachings herein.

FIGS. 8O-8P show examples of a fundus image and a visual representation of SHAP analysis, respectively, for an eye found to have an ocular lesion by using a detection model comprising the DenseNet169 model, trained via transfer learning for detection of ocular lesions and SHAP analysis according to the teachings herein.

FIGS. 9A-9B show confusion matrices presenting the results of choroidal nevus binary classification using deep transfer learning using the InceptionV3 model during a training phase and a validation phase, respectively.

FIGS. 9C-9D show a loss graph and an accuracy graph, respectively, of results of a choroidal nevus binary classification using deep transfer learning with the InceptionV3 model.

FIGS. 10A-10B show confusion matrices presenting the results of choroidal nevus binary classification using deep transfer learning using the Xception model during a training phase and a validation phase, respectively.

FIGS. 10C-10D show a loss graph and an accuracy graph, respectively, of results of a choroidal nevus binary classification using deep transfer learning with the Xception model.

FIGS. 11A-11B show confusion matrices presenting the results of choroidal nevus binary classification using deep transfer learning using the DenseNet121 model during a training phase and a validation phase, respectively.

FIGS. 11C-11D show a loss graph and an accuracy graph, respectively, of results of a choroidal nevus binary classification using deep transfer learning with the Inception DenseNet121 model.

FIGS. 12A-12B show confusion matrices presenting the results of choroidal nevus binary classification using deep transfer learning using the DenseNet169 model during a training phase and a validation phase, respectively.

FIGS. 12C-12D show a loss graph and an accuracy graph, respectively, of results of a choroidal nevus binary classification using deep transfer learning with the DenseNet169 model.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that, as used herein, the wording “and/or” is intended to represent an inclusive-or. That is, “X and/or Y” is intended to mean X or Y or both, for example. As a further example, “X, Y, and/or Z” and “any operable combination of X, Y and Z” is intended to mean X or Y or Z or any combination thereof that is operable.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments that are operable and have utility, unless otherwise specified to be not combinable or to be alternative options.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

At least a portion of the example embodiments of the systems or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and non-volatile memory). These devices may also have at least one input device (e.g., a touchscreen, and the like) and at least one output device (e.g., a display screen, a printer, a wireless radio, and the like) depending on the nature of the device.

It should also be noted that some elements that are used to implement at least part of the embodiments described herein may be implemented via software that is written in a high-level procedural language such as object-oriented programming. The program code may be written in JAVA, PYTHON, C, C++, Javascript or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage medium (e.g., a computer readable medium such as, but not limited to, ROM, flash memory, magnetic disk, optical disc) or a device that is readable by a programmable device. The software program code, when read by the programmable device, configures the programmable device to operate in a new, specific, and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, digital versatile disks (DVD), tapes, chips, and magnetic, optical or electronic storage. In alternative embodiments, the medium may be transitory in nature such as, but not limited to, wire-line transmissions, satellite transmissions, internet transmissions (e.g., downloads), media, digital or analog signals, and the like. The computer useable instructions may also be in various formats, including compiled or non-compiled code.

Accordingly, any module, unit, component, server, computer, terminal or device described herein that executes software instructions may include or otherwise have access to computer readable media such as storage media, computer storage media, or data storage devices (removable and/or non-removable) such as, for example, magnetic disks, optical disks, or tape. Computer storage media may include volatile or non-volatile, removable or non-removable media implemented in any method or technology for storage of information, such as computer readable instructions, data structures, program modules, or other data. Examples of computer storage media include RAM, ROM, EEPROM, flash memory or other memory technology, CD-ROM, DVD or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

It should be understood that use of the term “ocular lesion” herein is meant to cover any pathology visible on a fundus image that may be or simulate a benign or malignant mass.

It should be understood that the term “patient” generally means a person (aka human or individual) of any age such as a baby, child, teen, adult or senior.

As described herein, “medical images”, “image data”, or “images” refers to image data collected by image acquisition devices. The images are visual representations of an area of body anatomy that may be used for various purposes such as clinical analysis, diagnosis and/or medical interventions, commonly referred to as radiology. These images can be captured using fundus photography (aka fundus imaging). Fundus images show the inside, back surface of the eye, comprised of the retina, macula, optic disc, fovea, and blood vessels and may be photographed/imaged through the pupil using specialized microscopes with a camera attachment.

In medical data processing systems, image data and image metadata are typically stored in Digital Imaging and Communications in Medicine (DICOM) format. The DICOM image metadata typically includes information about how the medical image data was acquired, such as scanner data, anatomy data (e.g., the body regions imaged), image spatial data (e.g., spatial information about pixels and slice spacings), patient identifier data, image identifier data, acquisition date, acquisition time, etc.

A choroidal nevus is a lesion on the back of the eye that occurs in 0.2%-30% of adults. In most cases, choroidal nevi are harmless. However, an estimated 0.2% of choroidal nevi develop into UM, the most common form of intraocular cancer. If left untreated, UM can lead to vision loss, metastasis, or death. Identifying choroidal nevi can allow for UM screening, monitoring benign lesions, referring potentially malignant lesions, and treating potentially harmful lesions.

Currently, choroidal nevi are identified by healthcare practitioners using fundus photography as a diagnostic image. Color fundus photography provides an image representation of the condition of the eye's interior surface, determined by the reflected red, green, and blue wavebands (collectively referred to as RGB). Healthcare practitioners review fundus images and exercise clinical judgement to identify whether an ocular lesion is present in the fundus image.

However, identification of choroidal nevi can be challenging for healthcare providers, especially when the lesion is small. This issue is important since even small choroidal nevi can develop into UM. A further challenge exists in determining whether a small lesion is a benign nevus or a malignant tumor that should be treated expeditiously. These challenges lead to false negative and false positive diagnoses is. Typically, preliminary identification of choroidal nevi is performed by primary eye care providers, including optometrists, ophthalmologists and some general practitioners. When a choroidal nevus is suspected to be a malignant tumor, a referral to an ocular oncologist is typically provided to the patient. Malignant tumors that are not diagnosed can lead to severe complications. In addition, false positives can lead to excess referrals, causing healthcare resources to be used inefficiently.

Described herein are various example embodiments of devices, systems and methods that may be used for detecting ocular lesions. The various example embodiments disclosed herein generally use a detection model to identify whether a lesion that may be indicative of an ocular pathology is present in a fundus image. In at least one embodiment, the ocular lesion can be a choroidal nevus or a UM.

In at least one example embodiment described in accordance with the teachings herein, an ocular lesion detection system may be used to identify ocular lesions in medical image data (e.g., fundus images). The medical images can correspond to images acquired using conventional, existing techniques. It should be known that conventionally, these medical images are currently manually analyzed by healthcare providers. However, in accordance with the teachings herein, the images are processed in a new way leading to objective detection of ocular lesions.

The various embodiments described herein can assist primary eye care providers such as optometrists, general ophthalmologists and, in some cases, general practitioners in identifying and diagnosing ocular lesions. In at least one embodiment, recommendations, including referral and suggested treatments, can be made to the user (e.g., primary eye care provider) to assist the user in their ocular health assessments.

As described, the ocular lesion detection system can identify ocular lesions using a detection model. In at least one embodiment, the detection model can include a machine-learning model. The machine-learning model may be a supervised machine-learning model that is trained using labelled datasets. In the various embodiments described herein, the detection model can determine the presence or absence of lesions in ocular images.

As described, some lesions can be benign, while others can be malignant and correspond to malignant tumors. Accordingly, in at least one embodiment, the ocular lesion detection system can determine the risk of a lesion being a malignant tumor and provide an output (e.g., probability of malignancy, recommendation, suggested treatment) based on the lesion being determined to be a malignant tumor.

In at least one example embodiment, the ocular lesion detection system can be configured to display an output to assist the user (e.g., primary eye care provider) in diagnosing an ocular lesion and/or in assessing its potential malignancy. In at least one embodiment, annotated images and/or images showing the lesions can be presented to the user (“presented” or “presenting” meaning output on a display of an electronic device). The various embodiments described herein can be configured to present an output of a detection model for identifying and locating ocular lesions in a manner that is more easily interpretable. In at least one embodiment, a visual representation explaining the results of the detection model can be presented.

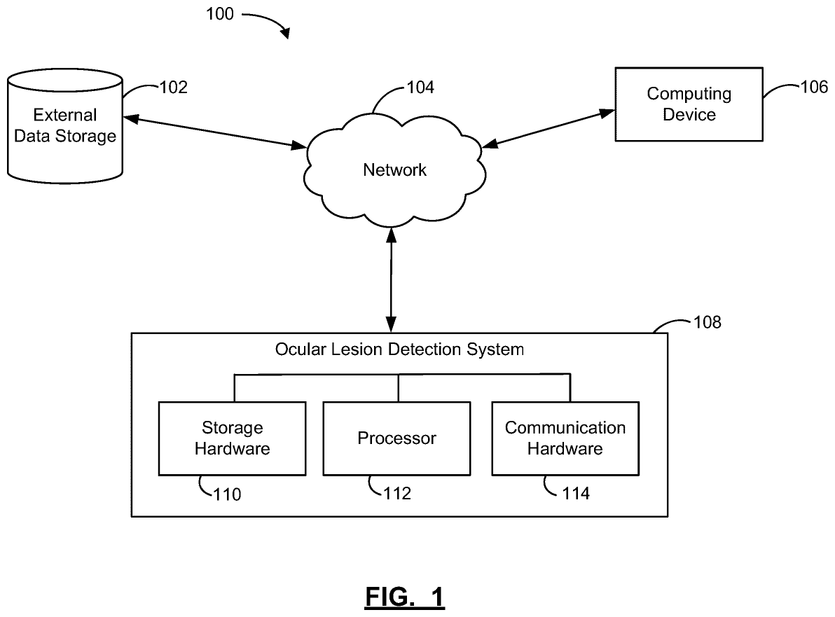

Referring now to FIG. 1, shown therein is a block diagram 100 of an example embodiment of a system 108 that can be configured for detecting ocular lesions that can be indicative of an ocular pathology. The system 108 can be in communication with an external data storage 102 and a computing device 106 via a communication network 104. Although only one computing device 106 is shown in FIG. 1, the ocular lesion detection system 108 may be in communication with a greater number of computing devices 106. The ocular lesion detection system 108 can communicate with the computing device(s) 106 via the network 104. The network 104 can be a local network or a wide geographic area network. The network 104 may be wired or wireless. In at least one embodiment, the ocular lesion detection system 108 or one or more components of the ocular lesion detection system 108 may be implemented by a computing device such as, but not limited to, a fundus photography device having computing capabilities, a desktop computer, a laptop, a tablet, a notepad or a smart phone, for example.

The ocular lesion detection system 108 includes storage hardware 110, processor 112, and communication hardware 114. The ocular lesion detection system 108 can be implemented on a computer server or more than one computer server distributed over a wide geographic area and connected via the network 104. Accordingly, the system 108 may be implemented using one or more processors (i.e., at least one processor). The storage hardware 110, the processor 112 and the communication hardware 114 may be combined into a fewer number of components or may be separated into further components.

The processor 112 can be implemented with any suitable processor, controller, digital signal processor, graphics processing unit, application specific integrated circuits (ASICs), and/or field programmable gate arrays (FPGAs) that can provide sufficient processing power for the configuration, purposes, and requirements of the ocular lesion detection system 108. In at least one embodiment, more than one processor may be used, with each processor being configured to perform different dedicated tasks.

The communication hardware 114 can include any interface that enables communication between the ocular lesion detection system 108 and one or more users or one or more devices that may be remote from the ocular lesion detection system 108. For example, the communication hardware 114 can receive inputs (e.g., images) from the computing device 106 and store the inputs in the storage hardware 110 or external data storage 102. The processor 112 can then process the images according to the methods described herein. The communication hardware 114 can then transmit results obtained by the ocular lesion detection system 108 to the one or more users or the one or more devices.

In at least one embodiment, the communication hardware 114 can include at least one communication port including one or more serial ports, one or more parallel ports and/or one or more USB port for electronic connection to various input and output devices.

In at least one embodiment, the communication hardware 114 can include a network interface to communicate with other devices via where the interface may be one or more of an Internet, Local Area Network (LAN), Ethernet, Firewire, modem, fiber, or digital subscriber line connection.

In at least one embodiment, the communication hardware 114 can include one or more radios that communicate utilizing CDMA, GSM, GPRS or Bluetooth protocol according to standards such as IEEE 802.11a, 802.11b, 802.11g, or 802.11n. The communication unit 114 can be used by the electronic device 102 to communicate with other devices or computers. The communication unit 114 can be a radio that communicates utilizing CDMA, GSM, GPRS or Bluetooth protocol according to standards such as IEEE 802.11a, 802.11b, 802.11g, or 802.11n.

In at least one embodiment, the communication hardware 114 can include input hardware to receive input data from various input devices, such as a mouse, a keyboard, a touch screen, a thumbwheel, a trackpad, a track-ball, a card-reader, and/or voice recognition software, and the like depending on the requirements and implementation of the ocular lesion detection system 108.

In at least one embodiment, the communication hardware 114 can include output hardware, which may include, for example, a display device, a printer and/or a speaker. In some cases, the display device may be used to provide one or more graphic user interfaces (GUIs) through an Application Programming Interface. A user may then interact with the one or more GUIs via the user interface for configuring the system 100 to operate in a certain fashion and/or providing input data as well as for viewing outputs that are generated by the ocular lesion detection system 108.

In various embodiments, the communication hardware 114 includes various combinations of communication ports, network interfaces, radios, input hardware and output hardware.

The storage hardware 110 can include RAM, ROM, one or more hard drives, one or more flash drives or some other suitable data storage elements such as disk drives. The storage hardware 110 can include one or more databases for storing inputs (e.g., images), including training inputs for the detection model, for storing the detection model itself, processed inputs, information relating to suggested treatments and results related to the inputs and training inputs (e.g., classification information related to images).

The external data storage 102 can store data similar to that of the storage hardware 110. The external data storage 102 can, in some embodiments, be used to store data that is less frequently used and/or older data. For example, the external data storage 102 can store previously assessed images. In some embodiments, the external data storage 102 can be a third-party data storage stored with input data for analysis by the ocular lesion detection system 108. The data stored in the external data storage 102 can be retrieved by the computing device 106 and/or the ocular lesion detection system 108 via the network 102.

Images described herein typically refer to fundus photographs. As will be described in further detail below, the ocular lesion detection system 108 can apply image preprocessing to the images such as, but not limited to, separation into RGB channels, cropping to remove the black portion of the fundus image, and normalizing the image to reduce light variations. In other embodiments, other preprocessing techniques may be used to improve signal-to-noise ratio and/or remove one or more artifacts as is known by those skilled in the art.

The computing device 106 can include any device capable of communicating with other devices through a network such as the network 102. A network device can couple to the network 102 through a wired or wireless connection. The computing device 106 can include a processor and memory and may be an electronic tablet device, a personal computer, a workstation, a server, a portable computer, a personal digital assistant, a laptop, a smart phone, a WAP phone, an interactive television, video display terminals, or portable electronic devices, or any combination of these. The computing device 106 can include a display, a user interface, a power supply unit and a communication unit. The display of the computing device 106 can be any suitable display that provides visual information depending on the configuration of the computing device 106. The display of the computing device 106 can display an output of the ocular lesion system 108 to a user of the computing device 106.

The network 104 can include any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, or others, including any combination of these, capable of interfacing with, and enabling communication between, the ocular lesion detection system 108, the external data storage 102, the computing device 106 or any other electronic devices (not shown).

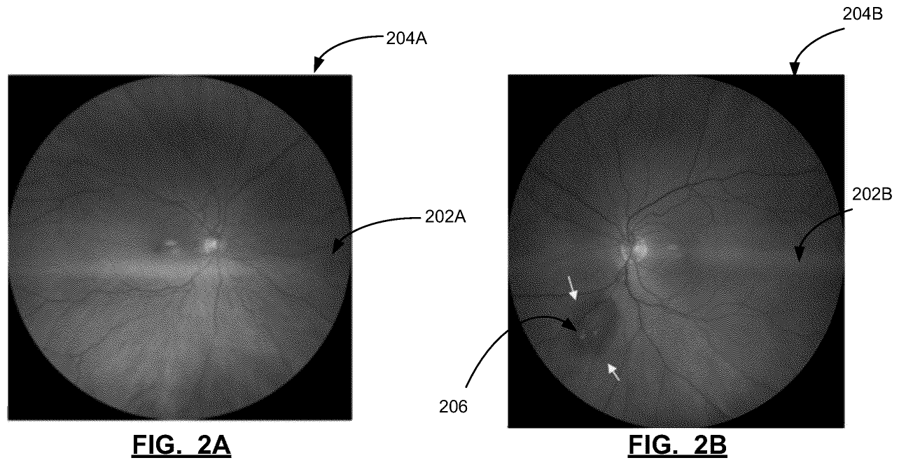

Reference is next made to FIGS. 2A, and 2B which show an example fundus photograph (aka a digital image) of an eye without an ocular lesion and an example fundus photograph of an eye with an ocular lesion 206. As shown, a fundus photograph is a diagnostic image of the fundus, the back surface of the eye. As shown, fundus photographs show the fundus 202 surrounded by a black portion 204. In some cases, as shown in FIG. 2B when the ocular lesion is large, it may be possible for a healthcare practitioner to identify lesions and determine its presence with the naked eye. However, in many cases, assessments by general healthcare practitioners can be unreliable. As described, the ocular lesion detection system 108 can be used to detect lesions such as lesion 206.

Referring now to FIG. 3, shown therein is a flowchart of an example embodiment of a method 300 for detecting ocular lesions. Method 300 can be performed by the processor 112 of the ocular lesion detection system 108 or another electronic device when executing software instructions that encodes the various steps and functionality of the method 300.

At step 302, the method 300 involves obtaining a fundus image of an eye of a patient such as an individual such as a human or an animal. The fundus image can, for example, be a digital image that is retrieved from a file stored in a data store, such as the storage hardware 110 or the external data storage 102. The fundus image can be an RGB image (i.e., an image containing red, green and blue channels). Alternatively, the fundus image may be obtained in real-time from a fundus photography device.

At step 304, the method 300 involves preprocessing the fundus image to obtain a processed fundus image. The preprocessing of the fundus image generally involves separating the RGB channels of the fundus image. In at least one embodiment, other preprocessing techniques may be used to improve signal-to-noise ratio and/or to remove artifacts. In embodiments where the fundus image is obtained in real time from a fundus photography device, the method 300 can involve preprocessing the fundus image to identify if artifacts likely to affect the ocular lesion detection system's 108 ability to detect that a lesion is present in the image obtained and instructing the fundus photography device to obtain another fundus image if such artifacts are present, or notifying the user to capture another fundus image.

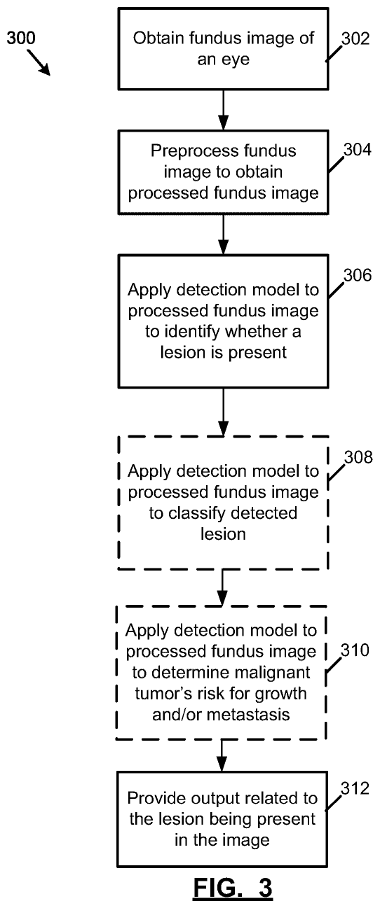

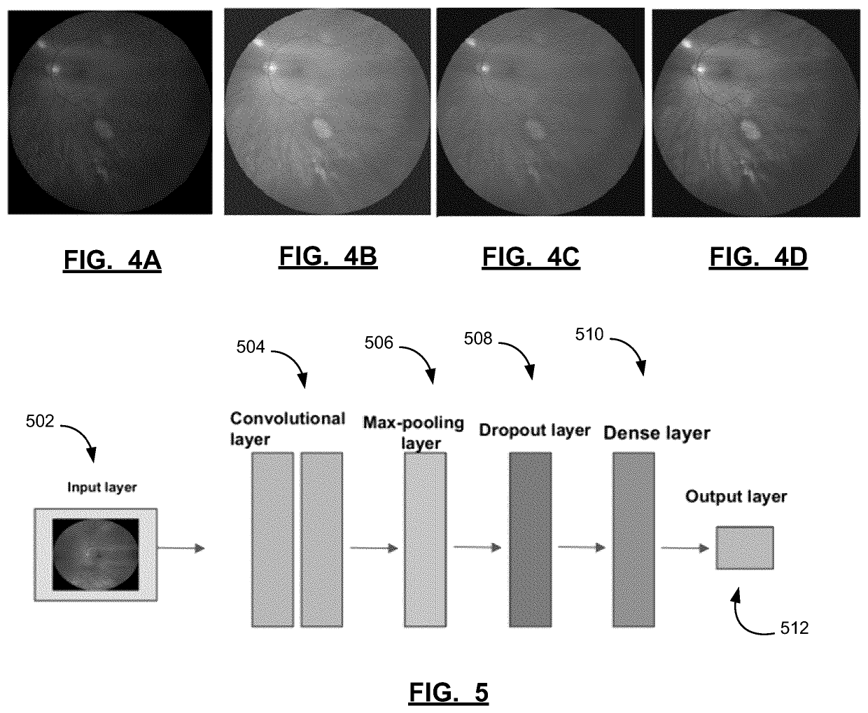

Reference is now briefly made to FIGS. 4A-4D, which show example fundus images. FIG. 4A shows an RGB image of a fundus image that can be obtained at step 302. FIG. 4B shows the fundus image of FIG. 4A with the green and red channels removed, leaving the blue channel only. FIG. 4C shows the fundus image of FIG. 4A with the blue and red channels removed, leaving the green channel only. FIG. 4D shows the fundus image of FIG. 4A with the green and blue channels removed, leaving the red channel only. The inventors have found that the green channel better represents the main features of a fundus image and, accordingly, that lesions are more accurately detected in processed fundus images showing only the green channel. Accordingly, at step 304, preprocessing the fundus image can involve isolating the green channel of the fundus image obtained at step 302.

In some embodiments, preprocessing the fundus image can also involve cropping the fundus image to remove the black area surrounding the fundus. As shown for example in FIGS. 2A-2B, fundus images show the fundus in a circle surrounded by black corners. Removing one or more black portions surrounding the fundus may allow the ocular lesion detection system 108 to only detect lesions in the portions of fundus images corresponding to the fundus. The black portion can be removed using any suitable image cropping technique, including an image cropping tool, for example the image cropping function in OpenCV, Pillow, and scikit-image or another suitable software program. These functions work by converting the image to an array and a tuple of slice objects representing the region of interest to be cropped. The slice objects define the range of rows and columns to keep from the original image. These functions then extract the specified region and returns the cropped image as a new array. Finally, the cropped image is saved in the original image format (e.g., PNG or JPG).

In some embodiments, preprocessing the fundus image can also involve normalizing the pixels of the fundus image. The preprocessing operation and parameters may be saved as a preprocessing object in the software programs that will be used to preprocess any testing fundus images. As will be described below, the detection model for detecting ocular lesions can be trained on a training dataset. The training dataset can include preprocessed images, including images normalized to account for variations, including variations in light and contrast and to account for noisy image regions in the fundus images in the training dataset. The fundus image received at step 302 can be preprocessed according to the detection model used. The fundus image can be preprocessed using any type of image processing tool for normalizing images, for example, the OpenCV, Pillow and scikit-image tools, or other suitable software programs, for further image processing and analysis as described herein.

In some embodiments, preprocessing the fundus image can also involve detecting artifacts such as eyelashes in the fundus image. In some embodiments, when a fundus image is determined to contain artifacts, the ocular lesion detection system 108 can generate a notification indicating that the fundus image is unsuitable for lesion detection or that the result obtained by the ocular lesion detection system 108 may be impacted by the presence of artifacts. In embodiments where the fundus image is obtained in real-time, the ocular detection system 108 may alternatively, or in addition thereto, automatically capture another fundus image. In some embodiments, when the artifacts are small, preprocessing the fundus can also involve removing or smoothing/blurring the detected artifacts, using various filters for removing or smoothing/blurring artifacts, such as smoothing, Gaussian, or Laplacian of Gaussian filters.

In some embodiments, preprocessing the fundus image can also involve determining if the fundus image is blurry and, in some cases, sharpening the blurry images. For example, some blurry images may be enhanced by unsharp masking, where a blurred image of the original images is created using a low-pass filter, such as a Gaussian filter. A mask representing the high-frequency details present in the original image is then generated by subtracting the blurred image from the original image. The mask is then amplified by a factor called the “sharpening amount.” This step enhances the high-frequency components, making the details more prominent. The amplified mask is then added back to the original image. This process effectively increases the contrast at edges and transitions, making the image appear sharper.

At step 306, the method 300 involves applying a detection model to the processed fundus image to determine whether a lesion is present in the fundus image. The detection model can be a model that is generated to detect ocular lesions in fundus images. The lesions detectable by the detection model can include choroidal nevi and UM. In at least one embodiment, determining whether a lesion is present in the fundus image additionally involves determining the size and location of the lesion. The detection model can be implemented using a deep learning machine-learning model such as a convolutional neural network (CNN), for example. Deep learning allows features to be learned directly from raw images in contrast to traditional machine learning models, which often require at least some features to be extracted manually. Traditional machine learning models, which require manually selecting features, can however still be used for analyzing the processed images to detect an ocular lesion For example, Support Vector Machine (SVM), a powerful classification algorithm that works by finding a hyperplane that best separates different classes in feature space can be used in at least one alternative embodiment. In identifying lesions, features extracted from segmented fundus images can include shape characteristics, texture information, and colour features. For instance, segmented regions' area, perimeter, eccentricity, and other morphological features can be used as inputs to an SVM classifier. SVM is particularly effective when the classes are well-separated, and the feature space is high-dimensional.

As another example, decision trees model may be used. Decision trees are intuitive models that partition the feature space into regions based on decision rules. These models are beneficial for capturing non-linear relationships between features and class labels. Decision trees can be employed to learn rules from features like shape descriptors, texture patterns, and pixel intensity statistics extracted from segmented regions when identifying ocular lesions. However, decision trees can be prone to overfitting, though overfitting can be mitigated by using ensemble methods such as Random Forests.

As described, traditional machine learning models such as SVM and decision trees, require selection of the most relevant and discriminative features for accurate classification, which can be challenging. Accordingly, embodiments which employ such machine learning models may use human expertise/experience in defining features, as described above, as domain knowledge is used to select clinically relevant features for ocular lesion detection.

In at least one of the embodiments described herein, the detection model can be a combination of models, for example, two or more of the models described above. In such embodiments, the detection model can be a model trained using ensemble learning and the result of the detection model can be a combination of the results of each of the models forming the detection model.

For example, in at least one embodiment, the detection model can be a CNN that operates as a binary classifier that can predict whether a fundus image contains a lesion or does not contain a lesion. Alternatively, in at least one other embodiment, the detection model can be a CNN that is a multiclass classifier. For example, the detection model can be a model that classifies the severity of an ocular lesion or any model that classifies its output into three or more categories. As another example, the detection model can be a model that, when an ocular lesion is determined to be a UM, classifies the UM in terms of its risk for growth or metastasis.

Referring briefly to FIG. 5, shown therein is a simplified diagram of a machine-learning model that can form a part of the detection model. As shown, the machine-learning model can be a CNN that takes as input image 502 (e.g., a fundus digital image) and feeds the input image 502 into a series of layers, including a convolutional layer 504, a max-pooling layer 506, a dropout layer 508 and a dense layer 510 to obtain an output 512. The output 512 can correspond to a result that may include a binary output or a multiclass output.

The detection model can be a model that is trained using a labelled (i.e., presence of lesion, absence of lesion) training dataset of fundus images.

The training dataset includes images that are preprocessed. For example, the fundus images in the training dataset can be converted into a format suitable for training the dataset (e.g., PNG or JPG). The images can additionally be resized and/or rescaled depending on, for example, the requirements of the detection model to be trained. For example, the color values of the pixels of the images can be scaled by a factor of 1/255 and the width (W), height (H) and channel (C) parameters of the images can be adjusted to a 150×150×3 (W×H×C). The training fundus images in the training dataset can additionally be preprocessed to remove the black portion surrounding the fundus in fundus images. The training dataset can additionally be pre-processed to isolate the red, green and blue channels of the fundus images. The inventors have found that the green channel better represents the main features of a fundus image and increases time efficiency in the data preprocessing step, and accordingly the training dataset can be preprocessed to isolate the green channel. The training dataset can additionally be preprocessed to enhance blurry images and remove from the training dataset any blurry images that cannot be enhanced to a suitable level. The process for enhancing blurry images can be similar to the process for enhancing a given fundus image, described at step 304. The training dataset can additionally be preprocessed to remove artifacts. The process for removing artifacts can be similar to the process for removing artifacts from a given fundus image, described at step 304. Alternatively, training images containing artifacts may be removed from the training dataset.

The training dataset can additionally be augmented using data augmentation techniques by applying various transformations to fundus images in the training dataset to increase the size of the dataset, introduce diversity and reduce overfitting during the training of neural networks. The training dataset can be augmented using any data augmentation technique suitable for augmenting a fundus image training dataset. For example, the ImageDataGenerator library in Keras can be used. Data augmentation techniques available in Keras include Rotation, Width and Height Shift, Shear Transformation, Zoom, Horizontal and Vertical Flips, Brightness Adjustment Channel Shift, or Normalization. The training may be done as is explained in the study below.

The training dataset can be used to train a model for detecting ocular lesions.

In at least one embodiment, the detection model can include a model that is improved by transfer learning. Transfer learning is a machine learning technique that involves using a model for solving a first task and trained on a first dataset (i.e., a base model) to improve a model trained on a second dataset, for solving a different, but related task. Transfer learning can be used to avoid overfitting, and reduce computing time and complexity. Instead of training a model from scratch, transfer learning allows knowledge learned by a pre-trained model on a large dataset (e.g., ImageNet) to be leveraged and adapted to a new task with a smaller dataset. Transfer learning can be used, when the training dataset is relatively small.

In at least one of the embodiments described herein, the base model can be any type of model that is trained for image analysis and/or object detection and that can be adapted for detecting ocular lesions in fundus images. For example, the base model can be the Inception V3 model, the Xception model, the DenseNet121 model or the DenseNet169 model. These four models are high-performing models that have been trained for image analysis and/or objection detection and that can be adapted for detecting ocular lesions in fundus images according to the teachings herein. The base model can be trained on fundus images and/or any other type of images. The implementation of these models may be done as explained in the study below. Other base models that can be used for transfer learning may include VGG16, VGG19, MobileNet, or EfficientNet. Other models having a suitable size may also be used, depending on the characteristics of the fundus image data of these models. Additionally, other models pre-trained on medical imaging datasets or other image datasets can also be used (image datasets containing any type of image). The base model, trained for image analysis and/or object detection is then further trained on a labelled set (i.e., presence of lesion, absence of lesion) of fundus images so that the model can detect lesions in fundus images. The use of transfer learning allows knowledge learned by the base model to be leveraged for ocular lesion detection.

In some embodiments, an optional step 308 may be performed, which involves the method 300 determining whether the lesion detected at step 306 is malignant. In some embodiments, step 308 involves determining whether the lesion detected at step 306 is a malignant tumor. In such embodiments involving step 308, the detection model is configured to compute a risk of malignancy of the lesion, or a risk of the lesion being a malignant tumor so that when the detection model is applied to a processed fundus image that includes a lesion, the output of the detection model includes an assessment of malignancy. For example, in some cases, the detection model can compute the risk of malignancy or the risk of the lesion being a malignant tumor based on one or more characteristics of the processed fundus image. Uveal melanoma, for example, may be characterized by one or more of the following characteristics: a thickness of >2 mm, subretinal fluid, an orange pigment, a margin within −3 mm of the optic disc, an ultrasonic hollowness and an absence of a halo. In at least one embodiment, the detection model can identify and extract at least some of these features from the fundus image. In such embodiments, the detection model may be trained to determine the risk of malignancy or the risk of a lesion being a malignant tumor and the detection model can compute the risk of malignancy based on the presence of one or more of these factors. This may be done by training the detection model to determine these risks. The risk of malignancy may then be confirmed through a biopsy and/or histopathological analysis.

As described, some ocular lesions can be benign while other ocular lesions can be malignant, for example malignant tumors. By distinguishing between benign lesions and malignant (incl. malignant tumors), appropriate treatment options can be suggested, recommended and/or pursued. Appropriate treatment options can be suggested, recommended and/or pursued by distinguishing between benign lesions and malignant lesions (incl. malignant tumors). Once a lesion is identified as being benign or a malignant tumor, healthcare providers can recommend and pursue the most suitable treatment options for the patient. For benign lesions, emphasis may be placed on monitoring the lesion and regular follow-ups to ensure that they do not develop into malignant lesions (incl. malignant tumors) or cause adverse effects on vision. In some cases, benign lesions may require treatment if they cause discomfort or impact the patient's quality of life.

On the other hand, malignant lesions (incl. malignant tumors), such as UM, require prompt and appropriate treatment. Treatment options for malignant lesions (incl. malignant tumors) may include surgical removal of the tumor or affected tissue, a common treatment approach for certain malignant tumors. In some cases, however, complete surgical resection may not be feasible due to the tumor's location. Treatment options can also include radiation therapy, to target and destroy cancerous cells. In the case of UM, techniques like plaque brachytherapy or proton beam therapy may be employed. For certain cases of UM, immunotherapy, to stimulate the body's immune system to attack cancer cells may also be used. Other treatment options may include targeted therapies, such as drugs targeting genetic mutations or signaling pathways in cancer cells, which are currently being explored for UM treatment. In some cases, enucleation (i.e., removal of the eye) may be considered a treatment option. The ability to distinguish between benign lesions and malignant lesions (incl. malignant tumors) accurately through appropriate diagnostic tools, such as the present ocular lesion detection system 108 can aid healthcare professionals in determining the best course of action for each patient. Early and accurate detection of malignancy can significantly impact treatment outcomes and patient prognosis.

In at least one embodiment, an optional step 310 may be performed, which involves the method 300 determining the severity of the malignant lesion identified at step 308. For example, step 310 can involve determining a malignant tumor's risk for growth and/or metastasis. A malignant lesion (incl. malignant tumor) can pose varying levels of risks to the ocular and general health of the individual. For example, a malignant tumor can pose varying levels of risk to the ocular and general health of the individual depending on its risk for growth. For example, a high severity lesion (incl. high-risk tumor) can be associated with a higher likelihood of metastasis and death. Determining the severity of a lesion (including determining the risk for growth of a tumor) can help healthcare providers identify appropriate treatment options and can help healthcare providers determine the treatment urgency so that highly severe lesions (e.g., high risk tumors) can be adequately prioritized. A tumor may be high-risk if it is associated with a high likelihood of growth and/or metastasis while a tumor may be a low-risk if it is associated with a low likelihood of growth and/or metastasis.

At step 312, the method involves providing an output related to a lesion being present in the fundus image.

In at least one embodiment, the output is a classification. The classification can be provided to a healthcare practitioner. For example, the classification can be displayed on a display such as a display of the computing device 106. The classification can be a binary classification (i.e., presence of lesion, absence of lesion). Alternatively, when a lesion is detected, the classification can compute a binary classification related to malignancy (i.e., malignant tumor, benign lesion). Alternatively, if a lesion has malignant potential, a multiclass classification can be computed indicating its potential risk of malignancy or risk for growth and/or metastasis (e.g., low risk, medium risk, high risk).

In at least one embodiment, the output can be a recommendation. The recommendation can be a referral recommendation. For example, the method 300 can involve recommending the patient affected by the lesion to consult a specialized healthcare provider, such as an ocular oncologist. Alternatively, or in addition thereto, the recommendation can be a suggested treatment. For example, the suggested treatment can involve performing enucleation, radiation therapy including brachytherapy or proton beam therapy, or immunotherapy. The suggested treatment can be provided to a healthcare provider (e.g., a primary eye care provider), for example, in combination with an indication that a lesion is present and/or the severity of the lesion (including the risk for growth and/or metastasis if the lesion detected is a potential malignant tumor). The recommendation can be displayed on a display, such as the display of computing device 106.

In embodiments where the detection model determines a risk of malignancy (incl. risk of growth and/or metastasis if the lesion is a malignant tumor) of the lesion detected, the method 300 can involve providing a treatment recommendation based on the risk of (incl. risk of growth) determined. For example, for a benign lesion, the treatment recommendation can consist of monitoring the development of the lesion to ensure that the lesion does not become malignant. For a malignant lesion (incl. malignant tumor) tumor, the treatment recommendation can involve a referral to a specialized healthcare provider, such as an ocular oncologist, for further assessment.

In embodiments where the detection model is configured to determine the severity of the ocular lesion (incl. risk of growth and/or metastasis if the ocular lesion detected is a potential malignant tumor) detected, the method 300 can involve providing a treatment recommendation based on the risk or the severity. The treatment recommendation can be a suggested treatment. For example, generally, a tumor being at high risk of growth and/or metastasis may require radiation therapy (e.g., brachytherapy), while a tumor being at low risk of growth and/or metastasis may require regular monitoring (i.e., active surveillance). The suggested treatment may also vary depending on the size and the location of the ocular lesion, as determined by the detection model.

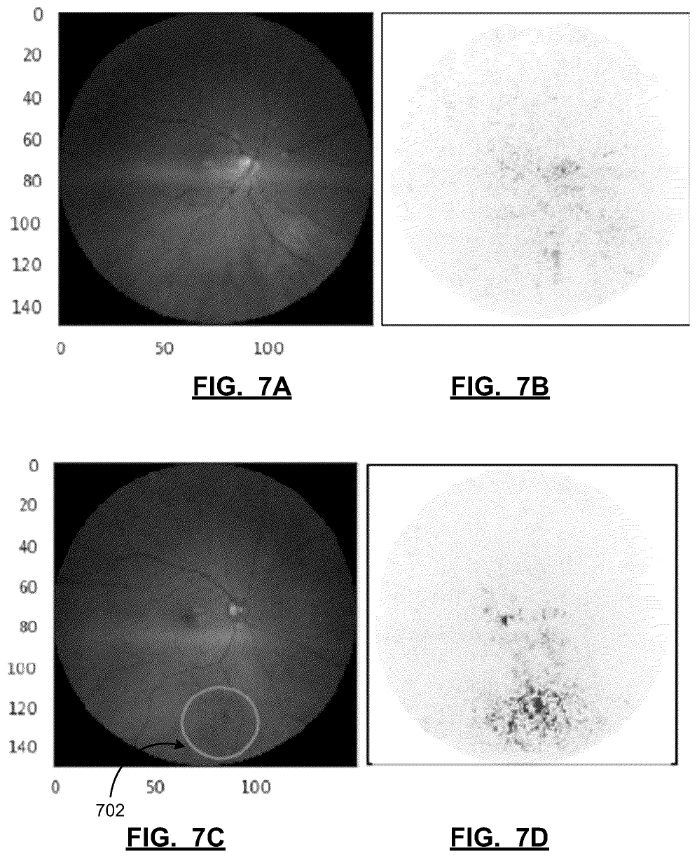

In some embodiments, when a lesion is detected, the output can include an annotated image of the fundus showing the lesion, as shown in FIG. 7C. For example, detected lesions can be circled as shown by circle 702 in FIG. 7C. In such cases, the method 300 involves annotating the fundus image to identify the lesion and displaying the annotated image. To annotate a fundus image, the method 300 can involve applying a mask over the region containing the lesion, outlining or adding a border around the mask to identify the lesion and removing the mask, so that the entire fundus image is shown, with the lesion identified. As described at step 306, in at least one embodiment, the detection model can determine the size and location of the lesion. In such embodiments, the mask can be selected based on the determined size of the lesion. The image can be displayed on a GUI of a display, for example, a display of the computing device 106. Annotating the fundus image to identify the lesion can help an eye care provider review the fundus image by drawing attention to the lesion.

In some cases, when a lesion is detected, the output can include a cropped version of the fundus image to isolate the lesion, which can be displayed on a display. Fundus images are typically large and have a high resolution and accordingly, displaying a cropped version of the fundus image can be more computationally efficient. In such cases, when the lesion is detected at step 306, the method 300 can involve detecting the contours of the lesion and automatically resizing the fundus image to remove the portions of the fundus image that are not affected by the lesion, and to only show the lesion, according to the contours detected. The lesion contour can be detected by automatically segmenting the lesion areas using segmentation models such as U-net segmentation models with attention. Alternatively, an active contour or snake algorithm can be used to isolate the contour of the lesion for presentation purposes. Alternatively, a mask having a similar size to the lesion can be applied over the fundus image and the fundus image may be cropped based on the size of the mask. As described at step 306, in at least one embodiment, the detection model can determine the size and location of the lesion. In such embodiments, the mask can be selected based on the determined size of the lesion. Using a mask to display only the affected portion of the fundus can assist eye care practitioners in reviewing fundus images and can save computational resources.

In at least one embodiment, the method 300 can involve applying a SHapley Additive exPlanations (SHAP) analysis to the detection model applied at 306. The SHAP analysis can be applied to a layer of the detection model, for example, the last fully connected layer of the model. SHAP is a method that is used to calculate the local importance of features of an input to determine the features of the input that contribute the most to the prediction of a detection model. SHAP can be applied to the detection model to identify areas of the fundus image that contribute the most to the determination of whether a lesion is present in the fundus image. A set of background data points is used to compute the SHAP values. These points serve as a reference distribution against which feature contributions are measured and are selected to represent the baseline or average case for the dataset. In this case, the background data points can come from fundus images without lesions. SHAP values can then be calculated for each fundus image by setting a baseline prediction by passing the fundus image through the detection model and obtaining a predicted probability for each class (e.g., binary—lesion/no lesion). To obtain these probabilities, background data points are sampled and added to the current image to form a dataset. The detection model's predictions are then obtained for the dataset. Then for each feature (neuron) in the final fully connected layer of the detection model, SHAP values are calculated by comparing the difference in predictions when the feature is active (using the current image) and when it's not active (using background data). By aggregating these differences across all features, the SHAP values for the current image can be obtained which indicate the impact of each feature on the model's prediction.

For interpretation, the positive SHAP values indicate that the presence of a feature pushes the model's prediction towards the positive class (i.e., ocular lesion) while negative values suggest the opposite effect (i.e., no ocular lesion). The magnitude of the SHAP value indicates the strength of the influence of a feature on the prediction. SHAP values can be visualized in various ways. For example, a SHAP summary plot can show the contribution of each feature for a specific prediction, as shown in FIG. 5. As another example, a force plot can be used to show how features push the prediction toward a certain class.

Applying SHAP to the final fully connected layer of a CNN model can provide insights into which features or regions in the fundus image contribute the most to the detection model's decision-making process. This interpretability is valuable for validating the model's predictions and better understanding its behaviour, especially in medical applications where transparency and accountability are advantageous.

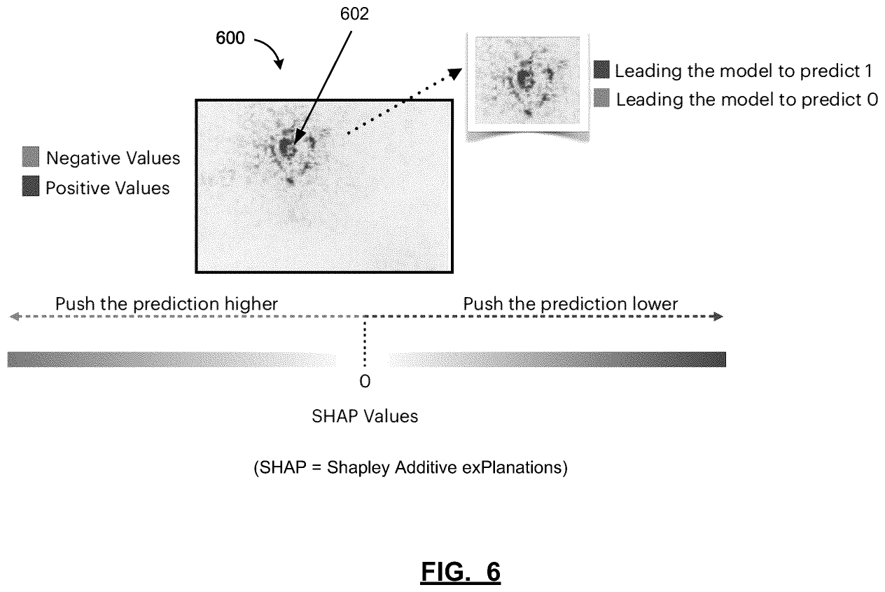

Referring briefly to FIG. 6, shown therein is a visual representation of the operation of a SHAP analysis. As shown, cluster 602 in the image 600 represents the area of the image that contributes the most to the prediction made by a detection model. As shown, each feature contributing to the prediction is assigned a value (positive or negative). The combination of the collection of values determines the prediction made by the detection model. In the example shown, the presence of a cluster of points having a positive value can be indicative of a lesion. SHAP computes the Shapley values from cooperative game theory to assign contributions to each feature (in this case, pixels in the fundus image) to the model's prediction. This allows understanding of how much each pixel in the input fundus image contributes to the final classification decision made by the model. Generally, in a binary detection model, the presence of a cluster of points having similar values can determine the outcome of the prediction.

Reference is now made to FIGS. 7A-7D. FIG. 7A shows a fundus image which, according to the detection model applied at step 306 does not contain lesions. As shown in FIG. 7B, which shows a visual representation of the SHAP analysis applied to the detection model for the fundus image of FIG. 7A, no cluster of points indicative of a lesion is present, indicating that the detection model did not detect lesions in the fundus image of FIG. 7A. A cluster of points in SHAP analysis usually indicates a region of the image that significantly influences the model's prediction. Conversely, the absence of a cluster of points suggests that the corresponding region has little impact on the model's decision. A cluster of points is typically correlated with the detection of a lesion by the detection model.

By contrast, FIGS. 7C-7D show a fundus image and a visual representation of a SHAP analysis where a lesion is present, respectively. As shown in FIG. 7D, the SHAP analysis shows a cluster of points around the lower portion of the image, indicating that the lower portion of the image contributed the most to the prediction made by the detection model.

In some cases, the visual representation of the SHAP analysis can be presented to the user of the ocular lesion detection system 108. For example, images such as those of FIGS. 7B and 7D can be displayed on a display, such as a display of computing device 106. Displaying a visual representation of the result of the SHAP analysis can assist healthcare providers (e.g., primary eye care providers) in identifying a region of interest in a fundus image. For example, in some cases, an annotated fundus image showing the lesion may not be displayed. In such cases, the visual representation of the result of the SHAP analysis can be correlated with the fundus image, so that the healthcare provider can identify a region of the fundus image requiring further analysis/assessment.

### Example 1: Study for Deep Learning-Based Detection and Classification of Uveal Melanoma Using Convolutional Neural Networks and SHAP Analysis

A study was performed that employed transfer learning techniques and four convolutional neural network (CNN)-based architectures (InceptionV3, Xception, DenseNet21 and DenseNet169) to detect UM and enhance the interpretation of diagnostic results. In the study, 854 RGB fundus images from two distinct datasets, representing the right and left eyes of 854 unique patients (427 lesioned and 427 non-lesioned) were manually gathered. Preprocessing steps, such as image conversion, resizing, and data augmentation, were performed before training and validating the classification results. The study utilized InceptionV3, Xception, DenseNet121, and DenseNet169 pre-trained models to improve the generalizability and performance of the results, evaluating each architecture on an external validation set.

Addressing the issue of interpretability in deep learning (DL) models to minimize the black-box problem, the study employed the SHapley Additive exPlanations (SHAP) analysis approach to identify regions of an eye image that contribute most to the prediction of choroidal nevus (CN). The performance results of the DL models revealed that DenseNet169 achieved the highest accuracy 89%, and lowest loss value 0.65%, for the binary classification of CN. The SHAP findings demonstrate that this method can serve as a tool for interpreting classification results by providing additional context information about individual sample images and facilitating a more comprehensive evaluation of binary classification in CN.

### Methods

The overall process implemented in the study included exploring the efficacy of machine learning for intraocular lesion detection and assessing the usefulness of this approach in identifying UM. The SHAP method is applied to interpret the classification results for CN.

### Dataset and Data Preparation

To develop and evaluate the deep learning algorithms for detecting choroidal nevus, the study utilized a dataset of color fundus images obtained from the diagnostic image repository of the Alberta Ocular Brachytherapy Program. These images were manually annotated by an orthoptics technician who had undergone appropriate training. The dataset consisted of a total of 606 fundus images, with 303 images labeled as having lesions and the other 303 labeled as not having lesions. The dataset size is 8.6 GB. The study acquired an additional dataset from the Wills Eye Hospital to further refine the deep learning models for choroidal nevus detection. This dataset was 258 MB in size and comprised fundus images of 248 patients collected by a medical expert from the clinic. Among these images, 124 were classified as non-lesion, while the remaining 124 were identified as having choroidal nevus.

Since the data source for monitoring the outcome in this research relies on image processing, the study implemented a series of image preprocessing techniques before using the data for the training process. These techniques include image conversion, resizing, and data augmentation.

Image Conversion: The quality of images used to train deep learning models impacts their performance [1], [2]. Therefore, the study initially gathered raw data from the Alberta Ocular Brachytherapy Clinic and Fight Eye Cancer in Digital Imaging and Communications in Medicine (DICOM) format [3]. To enhance the efficiency of the computations and ensure patient-level data stored in DICOM's metadata would not be exposed to third parties, the study converted the raw data to PNG format, extracting only the image portion.

Resizing: An input image consists of three parameters, including W×H×C, where W represents the width, H represents the height, and C denotes the number of channels in an image [4]. Each fundus image has three channels: red, blue, and green (RGB). In the preprocessing stage, the study assessed the prominence of the green channel compared to the other channels (blue, red). As noted, the inventors found that the green channel better represents the main features of a fundus image and increases time efficiency in the data preprocessing step [5]. Furthermore, to maintain consistency between the sizes of the input data and the data used in pre-trained models, the study adjusted the size of the input data to 150×150×3 for the InceptionV3, Xception, DenseNet121, and DenseNet169 models.

Augmentation: Additionally, to ensure more effective preprocessing in the Alberta Ocular Brachytherapy Program and the Wills Eye Hospital datasets with RGB images, the study rescaled the given images by a factor of 1/255 (i.e., range 0-1). Lastly, to implement data augmentation to increase the size of the training dataset, improve performance results, and enhance the generalizability of the detection architectures, the study employed the ImageDataGenerator library in Keras.

### Detection Models

Deep learning (DL) is a state-of-the-art machine learning technique paradigm that learns about data by passing it through hierarchical layers with non-linear processing [6]. DL models require extensive data to accomplish the feature engineering stage and avoid over-fitting when tuning hyperparameters during the training process. Therefore, to overcome the limitations of the small dataset (i.e., 854 images), the study employed transfer learning (TL) to transfer knowledge from large-scale pre-trained models, avoid overfitting, and reduce computing time and complexity [7]. The study used high-performing pre-trained deep transfer learning models (InceptionV3, Xception, DenseNet121, DenseNet169) [8].

Configurations: The study repeated the entire training/evaluation process of the binary classification task for batch size values of 8 and 32 and achieved similar performance results for both settings. However, since smaller batch size values yield higher performance with the categorical cross-entropy loss function [9], the study used a value of 8. The study plotted the accuracy and loss values over all epochs to ensure the model's results were not due to over-fitting. The study used Scikitlearn [10], Python 3.10 [11], and TensorFlow 2.9 [12] and trained and tested 9 GB of data comprising 854 fundus images on a MacBook M1 Pro 10-core CPU and GPU. The study used accuracy and loss values as measurements to improve the performance of the predictive models. A function in the Keras module was used to obtain these measurements during the training of the predictive models. to train and validate the prediction models developed in this study. Additionally, the study tried different values for the hyperparameters used to configure the detection models. The hyperparameters fine-tuned in this include learning rate (from 0 to 1), batch size (e.g., 8, 16, 32, 46), number of filters (e.g., 8, 16, 32, 64) and filter size (e.g., 3×3, 5×5). Adam optimizer and a learning rate of 0.0001 yielded the best performance and were used in the customized deep learning models [13], [14]. The study used weighting to address the data imbalance issue in the dataset [15]. The study employed the ImageNet pre-trained model to address this issue further add more context to the training process, boosting the learning pace and accuracy [16]. To prevent the weights of the pre-trained layers from being modified and minimize the computational time throughout each deep learning architecture, the study froze the weights of the feature extraction layers (i.e., pre-trained layers). It only modified the weights of the remaining layers to train the dataset [17]. Regarding the number of training epochs, the study experimentally defined 45 epochs to obtain the best training and validation set accuracy. However, the performance results showed no improvement after specific values [18]. Thus, to save computational resources, the study chose smaller values of epochs in this study (ranging from 50 to 150).

Transfer Learning Models: To classify fundus datasets for the detection of intraocular lesions in this study, four high-performing pre-trained transfer learning models were implemented: Inception-V3 [19], Xception [20], DenseNet121, and DenseNet169 [21]. Each model has a different-sized input layer defined in the Keras library [11]. In all pre-trained deep learning models, the first layer is followed by GlobalAveragePooling2D, a dropout layer with 20 to 40 percent, and a dense layer with 64 units applied for all four models, respectively. Before the output layer, the models have another dropout layer with the same rate of 20 to 40 percent. The study applied relu activation for the dropout layer, sigmoid activation for the output layer, and categorical cross-entropy for the loss function accordingly. The study used a kernel regularizer [22] in the dense layer to avoid overfitting and decrease the loss value. A ratio of 0.2 to 0.4 was applied to split the two datasets AOBP and FFEC for training 684 images and validating 170 images.

### Cohen's Kappa

To ensure consistency in the evaluations, the study employed Cohen's kappa [23] to assess the reliability of our four pre-trained transfer learning models. This method provided a practical framework for evaluating the DL models.

### SHAP Analysis

The study utilized the SHapley Additive exPlanations (SHAP) analysis model [8], [24]-[26] to determine the most significant features contributing to the classification results. This model employs game theory principles to calculate each player's contribution to a joint game. Specifically, in the context of deep learning and image processing, the SHAP values of each variable reveal the extent to which each feature (e.g., lesion size, location on an image) contributes to the detection outcome. Unlike traditional methods that assess feature importance across the entire dataset, SHAP analysis calculates local feature importance and assigns each feature a unique value for a specific prediction [27]. The study applied this technique to identify the important locations on fundus photos that indicate the presence of CN.

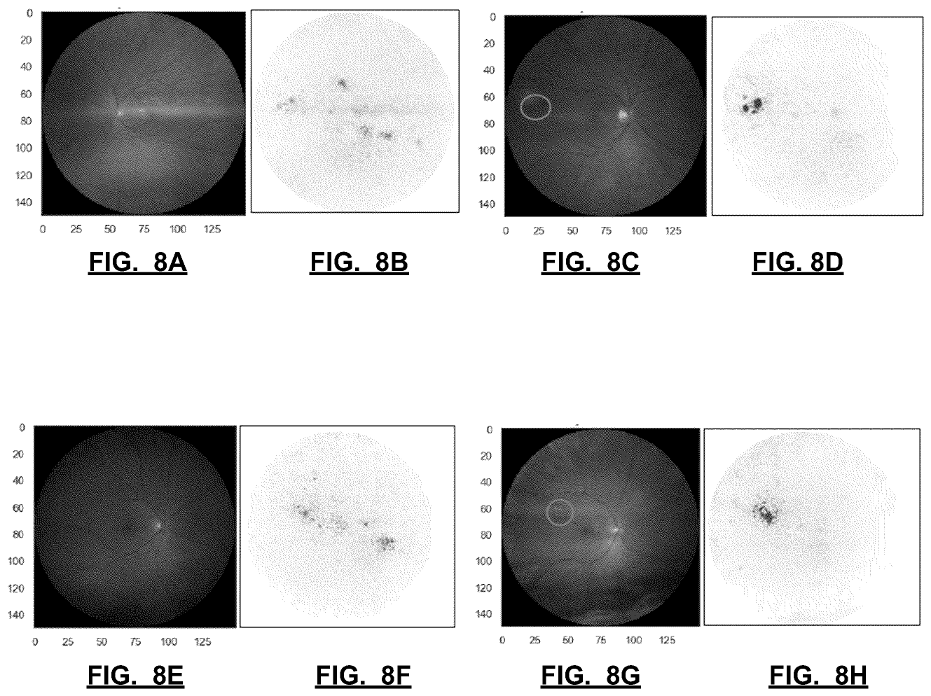

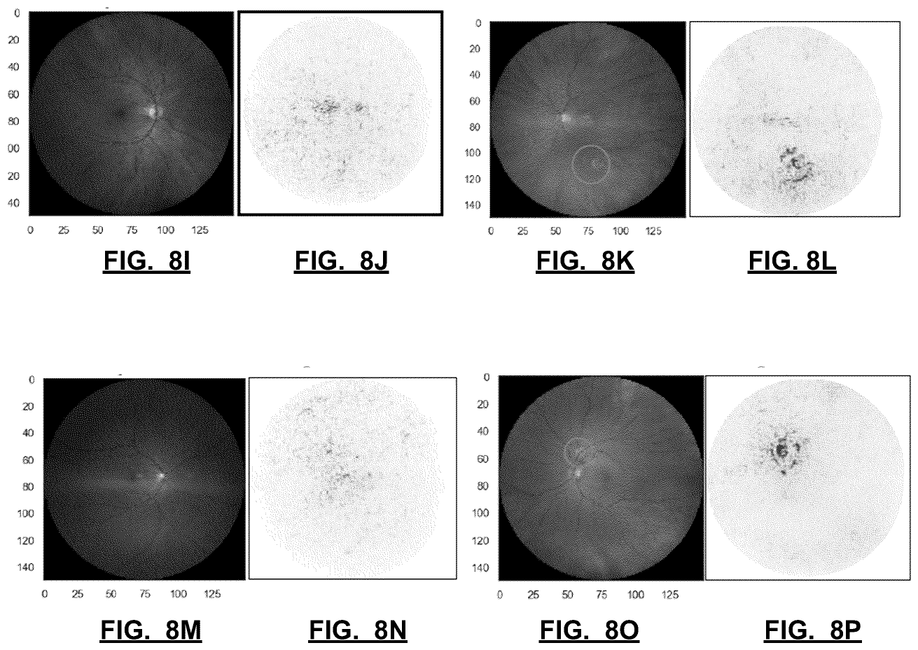

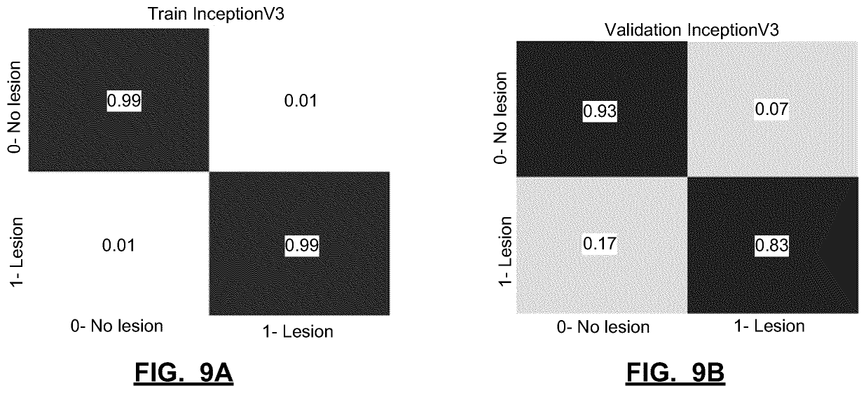

Referring briefly to FIGS. 8A-8P, shown therein are fundus images and visual representations of the results of SHAP analysis applied to the detection model. FIGS. 8B- and 8D correspond to SHAP results when the InceptionV3 model is used on the images of FIGS. 8A and 8C, respectively. FIGS. 8F and 8H correspond to SHAP results when the Xception model is used on the images of FIGS. 8E and 8G, respectively. FIGS. 8J and 8L correspond to SHAP results when the DenseNet121 model is used on the images of FIGS. 8I and 8K. FIGS. 8N and 8P correspond to SHAP results when the DenseNet169 model is used on the images of FIGS. 8M and 80.

### Results and Discussions

Table I shows the overall performance results of the models implemented in this study, including InceptionV3, Xception, DenseNet121, and DenseNet169. The highest validation accuracy of 89% with a loss value of 0.65 was achieved by the DenseNet169 model. InceptionV3 achieved an accuracy of 87% in validation with a loss value of 0.60, while DenseNet121 achieved an accuracy of 87% in validation with a loss value of 0.68. The Xception model achieved an accuracy of 84% with a loss value of 0.61.

TABLE I
Performance results for DL binary classification

| Method | Train acc | Val acc | Train loss | Val loss |
|---|---|---|---|---|
| InceptionV3 | 96% | 87% | 0.28 | 0.60 |
| Xception | 98% | 84% | 0.15 | 0.61 |
| DenseNet121 | 96% | 87% | 0.37 | 0.68 |
| DenseNet169 | 97% | 89% | 0.34 | 0.65 |

TABLE II
Kappa scores for DL binary classification

| Method | Train-Kappa | Val-Kappa | Complete-set-Kappa |
|---|---|---|---|
| InceptionV3 | 98% | 77% | 94% |
| Xception | 97% | 65% | 90% |
| DenseNet121 | 99% | 73% | 95% |
| DenseNet169 | 99% | 79% | 95% |

Table II shows the Kappa results for binary classification in the four CNN-based deep learning models. Among the four models, the DenseNet169 model achieved the best performance, with a validation Kappa of 79% and a complete-set Kappa of 95%. InceptionV3 achieved a validation Kappa of 77% and a complete-set Kappa of 94%, while DenseNet121 achieved a validation Kappa of 73% with a complete Kappa of 95%. The Xception model had a validation Kappa of 65% and a complete-set Kappa of 90%.

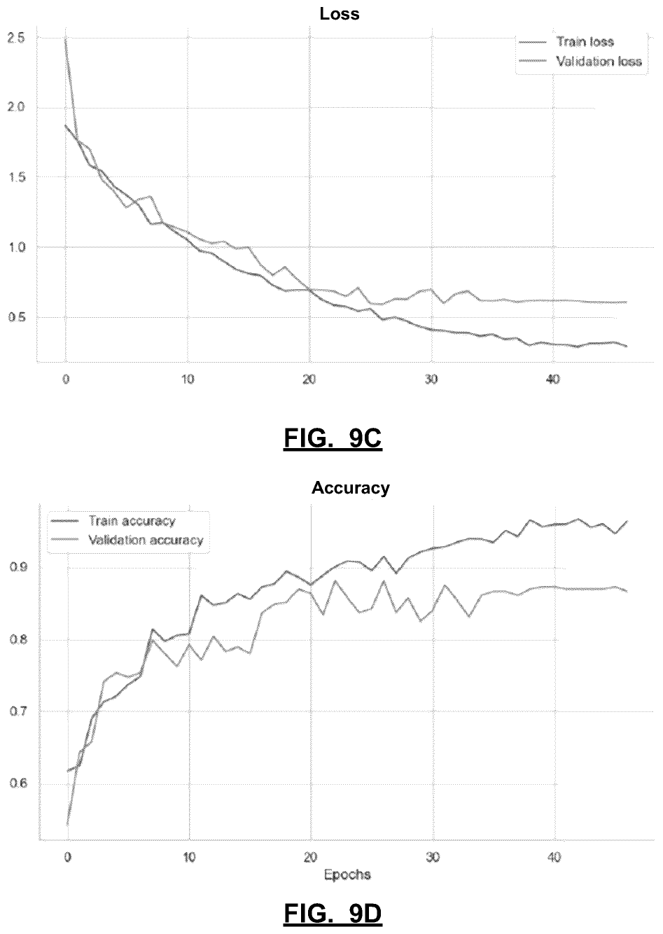

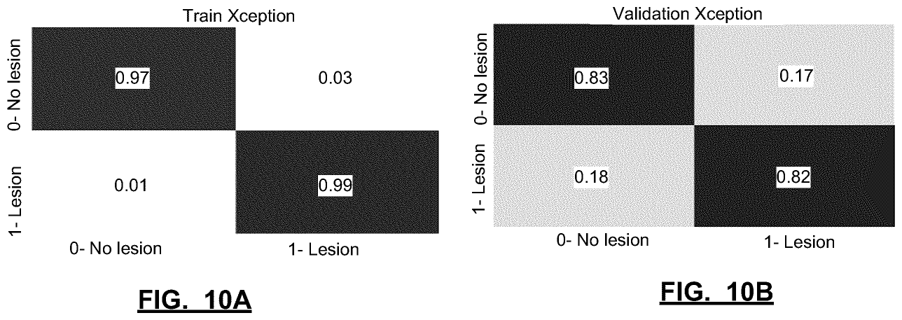

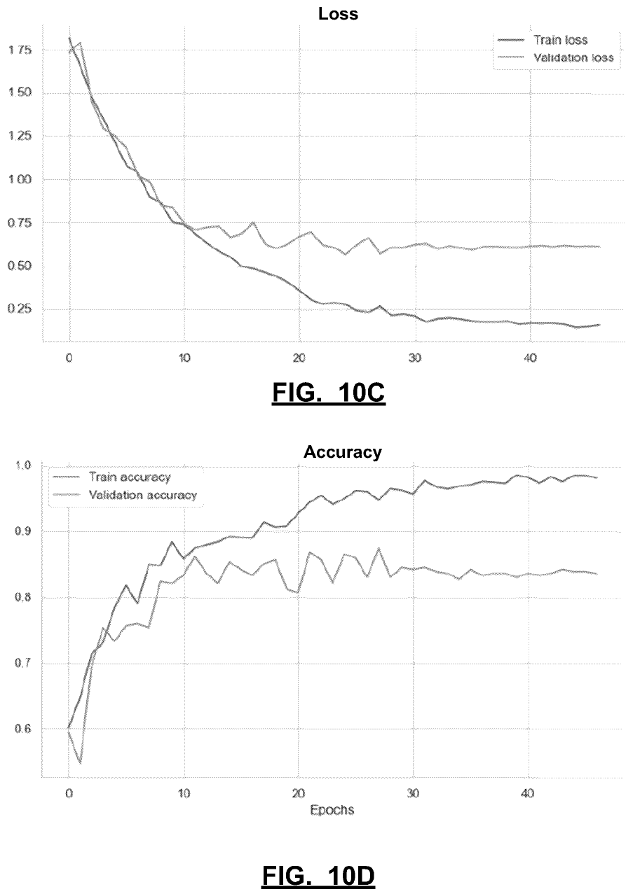

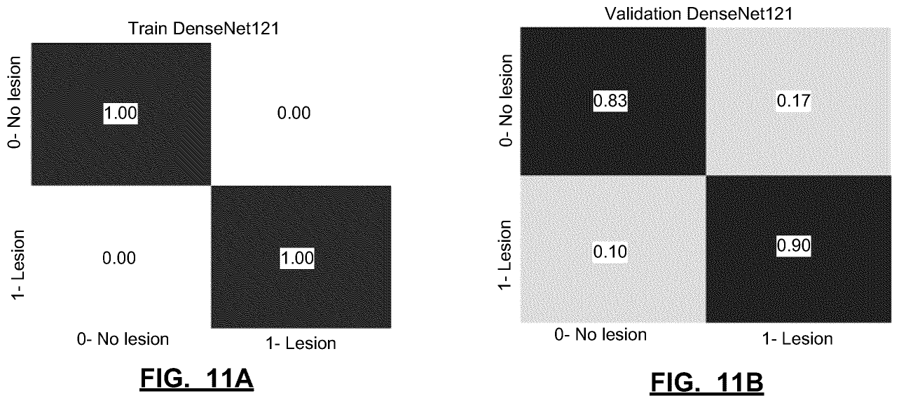

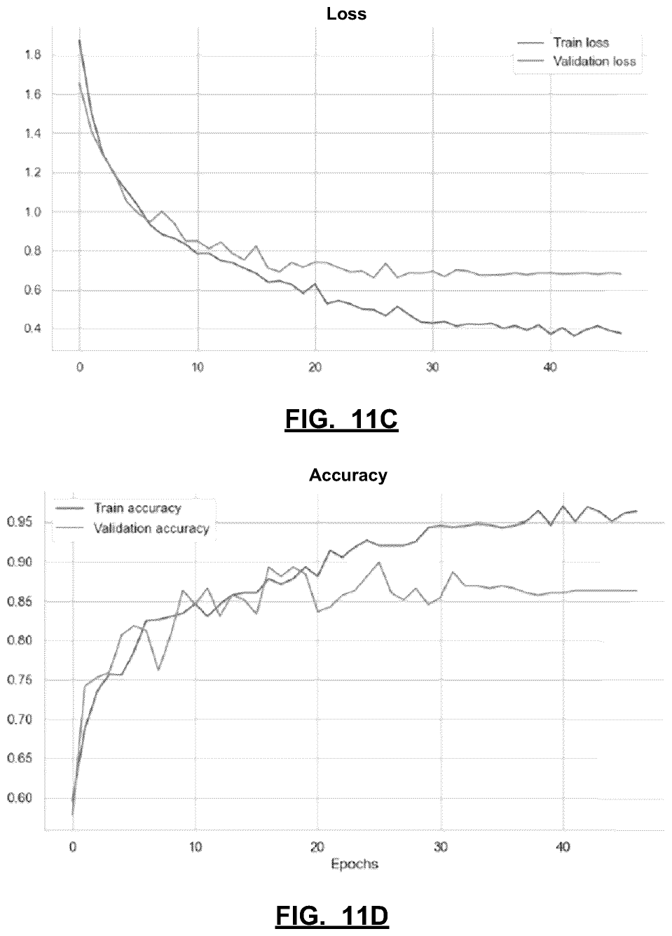

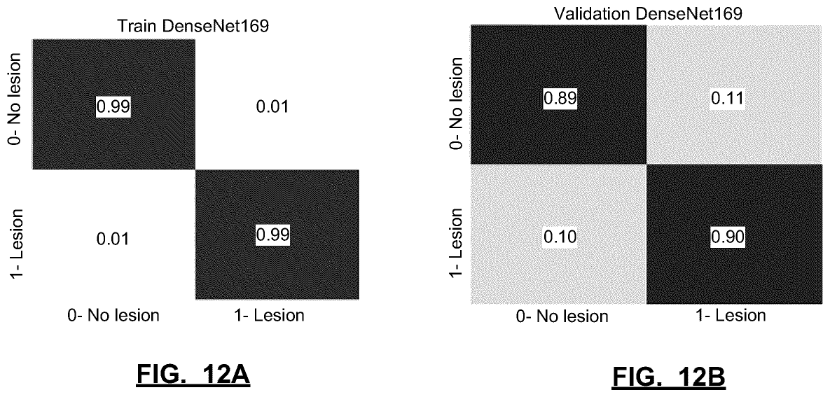

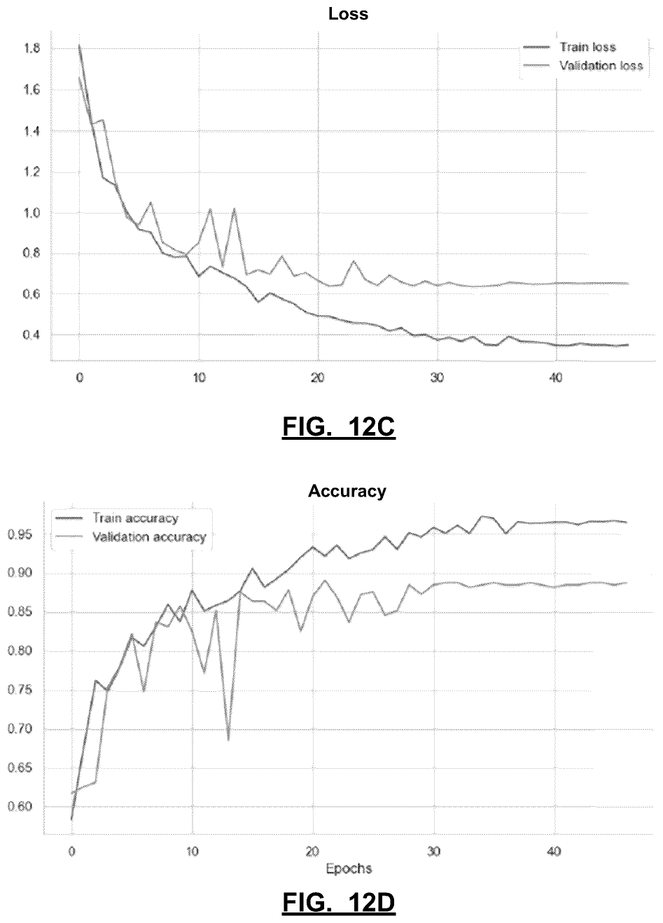

Reference is now made to FIGS. 9A-12D, which show confusion matrices and loss and accuracy plots for each of the four models studied. FIGS. 9A and 9B show the training and validation confusion matrices for the InceptionV3 model, respectively, indicating how well the InceptionV3 model performs on training and validation data. FIGS. 9C and 9D show the loss and accuracy plots, respectively, for the InceptionV3 model, showing the training progress. The decreasing loss and increasing accuracy over epochs suggest that the model is learning from the data. FIGS. 10A and 10B show the training and validation confusion matrices, respectively, for the Xception model. FIGS. 10C and 10D show the loss and accuracy plots, respectively, for the Xception model. The loss and accuracy plots in FIGS. 10C and 10D show how the model's loss decreases and accuracy increases during training. FIGS. 11A and 11B show the training and validation confusion matrices for the DenseNet121 model, respectively. FIGS. 11C and 11D show the loss and accuracy plots, for the DenseNet121 model, respectively. The loss and accuracy plots in FIGS. 10C and 10D show how the model's loss decreases and accuracy increases during training. FIGS. 12A and 12B show the training and validation confusion matrices for the DenseNet169 model, respectively. FIGS. 12C and 12D show the loss and accuracy plots for the DenseNet169 model, respectively. As shown, DenseNet169 achieved the highest validation accuracy (89%).

The higher validation accuracy suggests that the architecture's deeper layers more effectively capture complex features relevant to lesion detection. The loss and accuracy plots provide insight into convergence rates. A faster decrease in loss may indicate quicker learning though careful consideration is required to avoid overfitting.

Confusion matrices can be used to assess models. Models that tend to confuse certain classes may have specific weaknesses or strengths. Comparing relative performances can help identify the strengths and weaknesses of each model architecture in the context of ocular lesion detection.

### Key Findings

In this study, the performance of transfer learning models for binary classification of intraocular lesions and non-lesion cases using color fundus images was evaluated. In terms of ML efficacy, as shown in Table I, the study demonstrated that DL models can effectively detect intraocular lesions from color fundus images, achieving an accuracy of 89% and a loss of 0.65. This performance was achieved using DenseNet169, which outperformed other architectures tested in the study. These results suggest that deep learning models can be a valuable tool in aiding clinicians in the detection of intraocular lesions.

Regarding model interpretability, the study employed SHAP analysis to gain insights into the key features that contribute to the binary classification of fundus images. The SHAP analysis results showed that certain regions of the fundus image are more indicative of the presence or absence of intraocular lesions. These results, as depicted in FIGS. 8A-8P, highlight the areas that contribute most to CN detection in the transfer learning models. This information can aid clinicians in their decision-making process, as it provides a deeper understanding of the features that influence the model's predictions.

Since two small-datasets containing 854 images were used to train and assess the deep-learning models, various pre-trained transfer learning models trained on larger datasets were employed to improve the models' accuracy and address the issues associated with the size of the dataset. Incorporating more data into the proposed architecture may further enhance the performance and generalizability of the results. Elevated hyperparameters were selected for the detection models to specify their configurations and to manage the pre-trained models' training and validation. For example, 45 epochs were chosen to prevent overfitting and reduce the loss curve. However, the inventors' findings for binary classification in intraocular fundus lesions indicate that a higher epoch does not consistently improve the final results across different deep-learning architectures.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology.

### REFERENCES

[1]H. Jeelani, J. Martin, F. Vasquez, M. Salerno, and D. S. Weller, “Image quality affects deep learning reconstruction of mri,” in 2018 IEEE 15th International Symposium on Biomedical Imaging (ISBI 2018), 2018, pp. 357-360.
[2]J. Kugelman, D. Alonso-Caneiro, S. A. Read, S. J. Vincent, F. K. Chen, and M. J. Collins, “Effect of altered oct image quality on deep learning boundary segmentation,” IEEE Access, vol. 8, pp. 43 537-43 553, 2020.
[3]N. E. M. Association et al., “Digital imaging and communication in medicine (dicom),” NEMA PS 3 Supplement 23 Structured Reporting, 1997.
[4]T. H. Rafi and R. M. Shubair, “A scaled-2d cnn for skin cancer diagnosis,” in 2021 IEEE Conference on Computational Intelligence in Bioinformatics and Computational Biology (CIBCB), IEEE, 2021, pp. 1-6.
[5]Y. Liang, L. He, C. Fan, F. Wang, and W. Li, “Preprocessing study of retinal image based on component extraction,” in 2008 ieee international symposium on it in medicine and education, IEEE, 2008, pp. 670-672.
[6]Y. LeCun, Y. Bengio, and G. Hinton, “Deep learning,” nature, vol. 521, no. 7553, pp. 436-444, 2015.
[7]S. Yadav, S. Das, R Murugan, et al., “Performance analysis of deep neural networks through transfer learning in retinal detachment diagnosis using fundus images,” S adhan a, vol. 47, no. 2, pp. 1-13, 2022.
[8]E. Shakeri, T. Crump, E. Weis, R. Souza, and B. Far, “Using shap analysis to detect areas contributing to diabetic retinopathy detection,” in 2022 IEEE 23rd International Conference on Information Reuse and Integration for Data Science (IRI), IEEE, 2022, pp. 166-171.
[9]R. Nirthika, S. Manivannan, and A. Ramanan, “Loss functions for optimizing kappa as the evaluation measure for classifying diabetic retinopathy and prostate cancer images,” in 2020 IEEE 15th International Conference on Industrial and Information Systems (ICIIS), IEEE, 2020, pp. 144-149.
[10]F. Pedregosa, G. Varoquaux, A. Gramfort, et al., “Scikit-learn: Machine learning in python,” the Journal of machine Learning research, vol. 12, pp. 2825-2830, 2011.
[11]F. Chollet et al., “Keras: The python deep learning library,” Astrophysics source code library, ascl-1806, 2018.
[12]M. Abadi, P. Barham, J. Chen, et al., “{Tensorflow}: A system for {largescale} machine learning,” in 12th USENIX symposium on operating systems design and implementation (OSDI 16), 2016, pp. 265-283.
[13]A. A. E. F. Elsharif and S. S. Abu-Naser, “Retina diseases diagnosis using deep learning,” International Journal of Academic Engineering Research (IJAER), vol. 6, no. 2, 2022.
[14]D. P. Kingma and J. Ba, “Adam: A method for stochastic optimization,” arXiv preprint arXiv:1412.6980, 2014.
[15]H. Guo and H. L. Viktor, “Learning from imbalanced data sets with boosting and data generation: The databoost-im approach,” ACM Sigkdd Explorations Newsletter, vol. 6, no. 1, pp. 30-39, 2004.
[16]A. Krizhevsky, I. Sutskever, and G. E. Hinton, “Imagenet classification with deep convolutional neural networks,” Communications of the ACM, vol. 60, no. 6, pp. 84-90, 2017.
[17]S. Hinterstoisser, V. Lepetit, P. Wohlhart, and K. Konolige, “On pre-trained image features and synthetic images for deep learning,” in Proceedings of the European Conference on Computer Vision (ECCV) Workshops, 2018, pp. 0-0.
[18]S. S. M. Sheet, T.-S. Tan, M. As'ari, W. H. W. Hitam, and J. S. Sia, “Retinal disease identification using upgraded clahe filter and transfer convolution neural network,” ICT Express, vol. 8, no. 1, pp. 142-150, 2022.
[19]C. Szegedy, V. Vanhoucke, S. Ioffe, J. Shlens, and Z. Wojna, “Rethinking the inception architecture for computer vision,” in Proceedings of the IEEE conference on computer vision and pattern recognition, 2016, pp. 2818-2826.
[20]F. Chollet, “Xception: Deep learning with depthwise separable convolutions,” in Proceedings of the IEEE conference on computer vision and pattern recognition, 2017, pp. 1251-1258.
[21]G. Huang, Z. Liu, L. Van Der Maaten, and K. Q. Weinberger, “Densely connected convolutional networks,” in Proceedings of the IEEE conference on computer vision and pattern recognition, 2017, pp. 4700-4708.
[22]C. F. G. D. Santos and J. P. Papa, “Avoiding overfitting: A survey on regularization methods for convolutional neural networks,” ACM Computing Surveys (CSUR), vol. 54, no. 10s, pp. 1-25, 2022.
[23]J. Carletta, “Assessing agreement on classification tasks: The kappa statistic,” arXiv preprint cmp-Ig/9602004, 1996.
[24]S. M. Lundberg and S.-I. Lee, “A unified approach to interpreting model predictions,” Advances in neural information processing systems, vol. 30, 2017.
[25]E. Shakeri, E. A. Mohammed, Z. S. HA, and B. Far, “Exploring features contributing to the early prediction of sepsis using machine learning,” in 2021 43rd Annual International Conference of the IEEE Engineering in Medicine & Biology Society (EMBC), IEEE, 2021, pp. 2472-2475.
[26]E. Shakeri, T. Crump, E. Weis, E. Mohammed, R. Souza, and B. Far, “Explaining eye diseases detected by machine learning using shap: A case study of diabetic retinopathy and choroidal nevus,” SN Computer Science, vol. 4, no. 5, p. 433, 2023.
[27]I. Covert, S. M. Lundberg, and S.-I. Lee, “Understanding global feature contributions with additive importance measures,” Advances in Neural Information Processing Systems, vol. 33, pp. 17 212-17 223, 2020.

1. A method for detecting ocular lesions indicative of an ocular pathology, the method comprising:
obtaining a fundus image of an eye of a patient;
preprocessing the fundus image to obtain a processed fundus image;
applying a detection model to the processed fundus image to identify whether a lesion is present in the fundus image, the detection model being configured for detecting ocular lesions in fundus images; and
providing an output based on an output result of the detection model, the output being related to the ocular lesion being present in the fundus image.

2. The method of claim 1, wherein when the detection model detects the ocular lesion is present, the detection model is further configured to determine a risk of the lesion being a malignant tumor based on one or more characteristics of the fundus image, and
the method further comprises, in response to determining the ocular lesion is a malignant tumor, providing a treatment recommendation based on a predicted risk of growth and/or metastasis, wherein the treatment recommendation includes any combination of a referral recommendation or a suggested treatment, wherein the suggested treatment includes any combination of regular monitoring, radiation therapy, immunotherapy, targeted therapy, or removal of the eye.

3. The method of claim 1, wherein the detection model comprises a trained machine learning model including a convolutional neural network (CNN) or a CNN trained via transfer learning.

4. The method of claim 3, wherein the CNN trained via transfer learning is one of an Inceptionv3 model, an Xception model, a DensetNet121 model or a DenseNet169 model.

5. The method of claim 1, further comprising:
applying a Shapley Additive explanations (SHAP) analysis to the detection model to determine a contribution of each feature of the fundus image to the output result of the detection model; and
displaying a visual representation of the SHAP analysis.

6. The method of claim 1, wherein when the detection model detects the ocular lesion is present the method further comprises:
annotating the fundus image to obtain an annotated fundus image identifying the ocular lesion; and
displaying the annotated fundus image.

7. The method of claim 1, wherein when the detection model detects the ocular lesion is present, the method further comprises:
resizing the fundus image to obtain an image of the ocular lesion; and
displaying the image of the ocular lesion.

8. The method of claim 1, wherein the ocular lesion is a choroidal nevus or a uveal melanoma (UM).

9. The method of claim 1, wherein preprocessing the fundus image comprises isolating a green channel of the fundus image.

10. The method of claim 9, wherein preprocessing the fundus image further comprises one or more of: cropping the fundus image to remove a black portion surrounding a fundus in the fundus image, normalizing the fundus image to reduce light variations and sharpening the fundus image to reduce blurriness.

11. A system for detecting ocular lesions indicative of an ocular pathology, the system comprising:
a database for storing fundus images;
a memory for storing software instructions for processing a fundus image; and
at least one processor in communication with the memory and the database, the at least one processor, upon executing the software instructions, being configured to:
obtain the fundus image of an eye of a patient from the database;
preprocess the fundus image to obtain a processed fundus image;
apply a detection model to the processed fundus image to identify whether an ocular lesion is present in the fundus image, the detection model being configured for detecting the ocular lesion in fundus images; and
provide an output based on an output result of the detection model, the output result being related to the ocular lesion being present in the fundus image.

12. The system of claim 11, wherein when the detection model detects the ocular lesion is present, the at least one processor is configured to:
use the detection model to determine a risk of the ocular lesion being a malignant tumor based on one or more characteristics of the fundus image; and
in response to determining the ocular lesion is malignant, provide a treatment recommendation based on a predicted risk of growth and/or metastasis, wherein the treatment recommendation includes any combination of a referral recommendation or a suggested treatment, wherein the suggested treatment includes any combination of regular monitoring, radiation therapy, immunotherapy, targeted therapy or removal of the eye.

13. The system of claim 11, wherein the detection model comprises a trained machine learning model including a convolutional neural network (CNN) or a CNN trained via transfer learning.

14. The system of claim 13, wherein the CNN trained via transfer learning is one of an Inceptionv3 model, an Xception model, a DensetNet121 model or a DenseNet169 model.

15. The system of claim 11, wherein the at least one processor is further configured to:
apply a Shapley Additive explanations (SHAP) analysis to the detection model to calculate a contribution of each feature of the fundus image to the output result of the detection model; and
display a visual representation of the SHAP analysis.

16. The system of claim 11, wherein when the detection model detects the ocular lesion is present, the at least one processor is configured to:
annotate the fundus image to obtain an annotated fundus image identifying the ocular lesion; and
display the annotated fundus image.

17. The system of claim 11, wherein when the detection model detects the ocular lesion is present, the at least one processor is configured to:
resize the fundus image to obtain an image of the ocular lesion; and
display the image of the ocular lesion.

18. The system of claim 11, wherein preprocessing the fundus image comprises isolating a green channel of the fundus image.

19. The system of claim 18, wherein preprocessing the fundus image further comprises one or more of: cropping the fundus image to remove a black portion surrounding a fundus in the fundus image, normalizing the fundus image to reduce light variations and sharpening the fundus image to reduce blurriness.

20. A non-transitory computer readable medium storing thereon software instructions, which when executed by at least one processor, configure the at least one processor for performing a method for detecting an ocular lesion indicative of an ocular pathology wherein the method is defined according to claim 1.
